(19) United States
(12) Patent Application Publication
(10) Pub. No.: US 2011/0046587 A1
(43) Pub. Date: Feb. 24, 2011

(54) ABSORBENT PADS AND METHOD OF MAKING SUCH PADS

(75) Inventor: Joseph Meir Meizelman, Zug (CH)

Correspondence Address: MARTIN D. MOYNIHAN d/b/a PRTSI, INC., P.O. BOX 16446, ARLINGTON, VA, 22215, US

(73) Assignee: Bright Technologies Corp. Inc., Zug (CH)

(21) Appl. No.: 12/989,957

(22) PCT Filed: Apr. 27, 2009
(86) PCT No.: PCT/IL2009/000447

§ 371 Date: Oct. 28, 2010

Related U.S. Application Data
(63) Continuation of application No. 12/149,159, filed on Apr. 28, 2008.

(51) Int. Cl. A61F 13/15; B32B 37/16

(52) U.S. Cl. 604/359; 156/276

(57) ABSTRACT

A method of making an absorbent pad, and the absorbent pad so made, including a porous under layer and an absorbent body including a pulverized pulp and a super-absorbent materials bonded to and between the porous layers; characterized in that the absorbent body is applied between the porous layers before bonding thereto by: applying a first layer of pulverized pulp to the porous under layer; applying a layer of the super-absorbent material over the first layer of pulverized pulp; and applying a second layer of the pulverized pulp over the super-absorbent material layers. One or more of the above layers also includes a deodorizer material which may include a deodorizer agent, to reduce odors, an anti-inflammatory agent to prevent skin erythema, and/or a perfume fragrance.

### RELATED APPLICATION

This application is a continuation-in-part of U.S. patent application Ser. No. 12/149,159, filed Apr. 28, 2008 and claims the priority date of that application.

### FIELD AND BACKGROUND OF THE INVENTION

The present invention relates to methods of making absorbent pads, and also to absorbent pads made by such methods.

Absorbent pads, sometimes called underpads, are used in a wide variety of applications, including: medical applications, for wound care and hospital underpads; feminine hygiene, such as sanitary napkins, panty shields; baby and adult diapers; food packaging, such as soak pads for meat and fish packaging; and industrial filtration, such as for kerosene, hydraulic oils, etc.

Absorbent pads usually include a porous under layer, a porous upper layer, and an absorbent body including a pulverized pulp and a super-absorbent material bonded to and between the two porous layers. The two porous layers are generally made of tissue paper, whereas the super-absorbent material included in the absorbent body between the two porous layers is generally of a water-insoluble hydrogel-forming polymer capable of absorbing large quantities of fluids, such as water and body wastes, and further capable of retaining such absorbed fluids under moderate pressure. A large number of such super-absorbent materials are well known and available, as described for example U.S. Pat. No. 4,654,039.

Many methods have been developed for making such absorbent pads as described, for example, in U.S. Pat. Nos. 3,692,622, 6,675,702, 6,675,703 and 7,025,198. However, efforts are continuously being made to improve the properties of such absorbent pads, as well as their method of manufacture. For example,

U.S. Pat. No. 7,025,198 discloses an absorbent pad with a controlled rate of wicking. This is done by coating the upper and lower layers of porous material with a hydrophilic composition, which wicks liquid exuded by the subject, food product, or other object, into the absorbent layer, and thereby presents a dryer feel to the surface of the absorbent pad contacted by the source of the liquid. In such a construction, the rate of wicking can be controlled by changing the amounts of hydrophilic coating present in the absorbent layers. Such absorbent pads, however, are relatively costly to produce.

In addition, the absorbent pads generally available today do not include deodorants, anti-bacterial compositions, or fragrances, and therefore their use is frequently unpleasant both to the user, as well as those in the immediate environs.

### OBJECT AND BRIEF SUMMARY OF THE PRESENT INVENTION

An object of the present invention is to provide a method of making an absorbent pad having improved properties in one or more of the foregoing respects over the absorbent pads that have heretofore been developed. Another object of the invention is to provide an improved construction of such absorbent pads.

According to one broad aspect of the present invention, there is provided a method of making an absorbent pad comprising a porous under layer, a porous upper layer, and an absorbent body including a pulverized pulp and a super-absorbent material bonded to and between the porous layers; characterized in that the absorbent body is applied between the porous layers before bonding thereto by: applying a first layer of pulverized pulp to the porous under layer; then applying a layer of the super-absorbent material over the first layer of pulverized pulp; and then applying a second layer of the pulverized pulp over the super-absorbent material layers.

As will be described more particularly below, such a method provides a number of advantages over the presently used method of mixing the super-absorbent material with the paper pulp before applying the mixture to serve as the absorbent body. Thus, one important advantage in the method briefly described above is that the super-absorbent material, which holds most of the absorbed liquid, is further spaced from the upper surface of the pad in contact with the subject's skin by the second layer of pulverized pulp, and thereby presents a drier feel to the subject. In addition, the super-absorbent material can be selectively applied only to selected regions (i.e., not to the whole surface) of the porous under layer, thereby minimizing the quantity of the super-absorbent material used, which is a relatively expensive part in the cost of making such absorbent pads.

According to another aspect of the present invention, there is provided a method of making an absorbent pad comprising a porous under layer; a porous upper layer; and an absorbent body bonded to and between the porous layers; characterized in that the absorbent body also includes a deodorant composition. The deodorant composition may be in the form of a powder, liquid or spray, which includes a deodorant agent to reduce odors, an anti-inflammatory agent to prevent skin erythema during the use of the absorbent pad by a person, and/or a perfumed fragrance to prevent a pleasant overall odor to the absorbent pad.

In the described preferred embodiments, the deodorant agent is talc powder, and the anti-inflammatory agent is zinc oxide or aloe vera extract. Preferably, the deodorant powder mixture is present from 5 to 45 percent by weight of the intermediate layer. Also in the described preferred embodiments, the deodorant agent is present from about 60 to 80 percent by weight of the deodorant mixture powder. The deodorant mixture powder may also include a perfume fragrance, e.g. from 1 to 3 percent by weight of the powder mixture, to mask perspiration and/or urine odors.

According to further features in the described preferred embodiments, the layers are bonded together by pressure applied by embossing rollers. One or more layers may also be bonded by glue. The intermediate layer preferably has an initial water content of over 5 percent, e.g., between 7-10 percent, before the layers are bonded together.

According to further features in the described preferred embodiments, each of the upper and lower porous layers is an unwoven tissue layer such as paper, or a porous plastic film, such as micro-perforated polyethylene film.

Another embodiment is described wherein the three-layer pad defines a core in which the lower porous layer is backed by a backing layer of a non-porous plastic film, such as polyethylene. In the latter embodiment, the absorbent pad also includes an overlying porous layer over the upper porous layer and pressure bonded to the backing layer along margins extending along the four sides of the core. The overlying porous layer is provided particularly to enhance the “feel”. It may also be an unwoven plastic layer or a porous plastic film.

According to another aspect of the present invention, there is provided an absorbent pad comprising a porous under layer, a porous upper layer, and an absorbent body including a pulverized pulp and a super-absorbent material bonded to and between the porous layers; characterized in that the absorbent body also includes a deodorant composition.

Further features and advantages of the invention will be apparent from the description below.

### BRIEF DESCRIPTION OF THE DRAWINGS

The invention is herein described, by way of example only, with reference to the accompanying drawings, wherein:

FIG. 1 is a block diagram schematically illustrating a production line for one method of making three-layer absorbent pads in accordance with the present invention;

FIG. 2 is a sectional view illustrating the absorbent pad made according to the method of FIG. 1;

FIG. 3 illustrates the embossing rollers in the block diagram of FIG. 1;

FIG. 4 is a side view illustrating the surface of one of the embossing rollers of FIG. 3;

FIG. 5 is an enlarged fragmentary view more particularly illustrating the surface of the embossing rollers;

FIG. 6 is a block diagram schematically illustrating another production line for a method for making five-layer absorbent pads in accordance with the present invention;

FIG. 7 is a sectional view illustrating the absorbent pad made in accordance with the method of FIG. 6;

FIG. 8 is a flow chart illustrating the sequence of operations in the production line of FIG. 1;

FIGS. 9-14 are flow charts corresponding to that of FIG. 8, but illustrating a number of variations in the sequence of operations illustrated in FIGS. 8 and 1;

FIG. 15 is a flow chart illustrating the sequence of operations in the production line of FIG. 6;

FIG. 16 is a flow chart similar to that of FIG. 15, but illustrating a variation in the sequence of operations thereof;

FIG. 17 is a plan view of one form of absorbent pad constructed in accordance with the present invention;

FIG. 18 is a transverse sectional view along line XVII-XVII of FIG. 17;

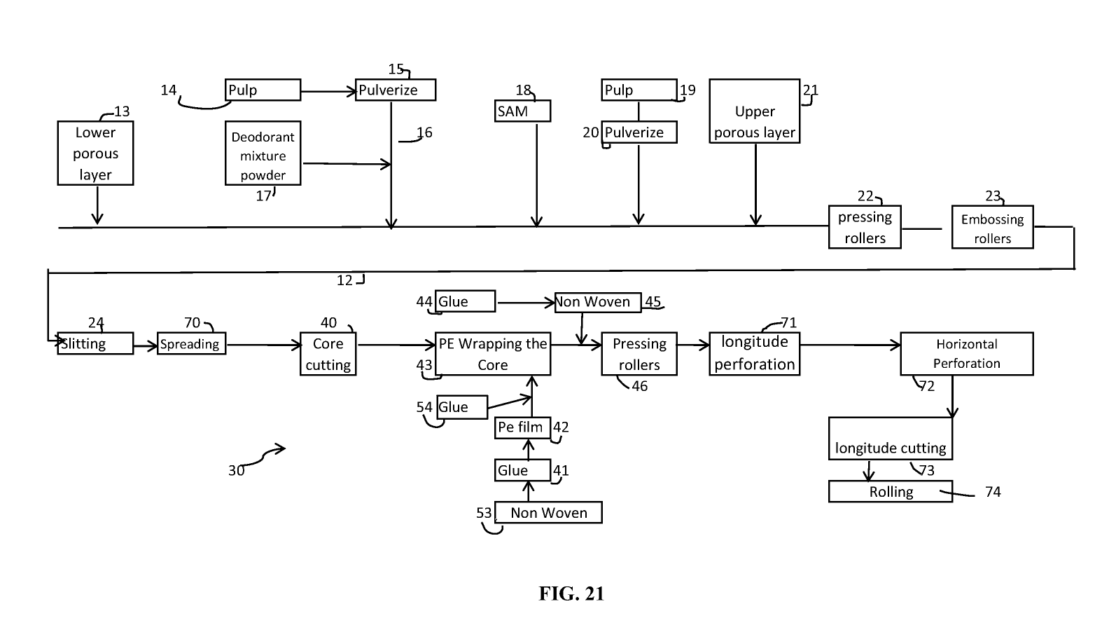

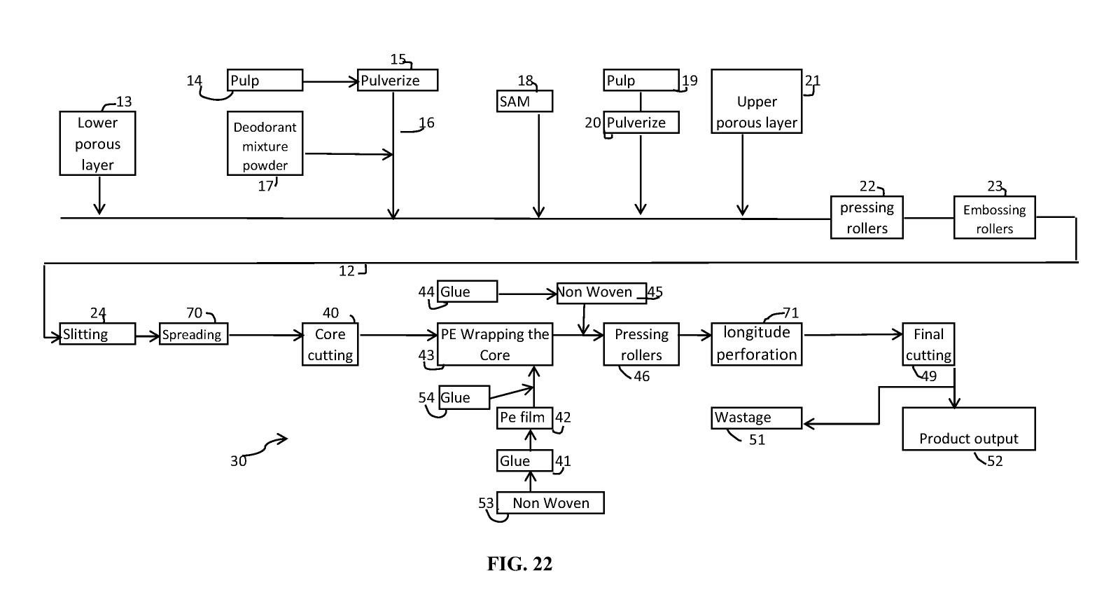

and FIGS. 19-22 illustrate various ways of producing the absorbent pad of FIGS. 17 and 18.

It is to be understood that the foregoing drawings, and the description below, are provided primarily for purposes of facilitating understanding the conceptual aspects of the invention and possible embodiments thereof, including what is presently considered to be a preferred embodiment. In the interest of clarity and brevity, no attempt is made to provide more details than necessary to enable one skilled in the art, using routine skill and design, to understand and practice the described invention. It is to be further understood that the embodiments described are for purposes of example only, and that the invention is capable of being embodied in other forms and applications than described herein.

### DESCRIPTION OF PREFERRED EMBODIMENTS

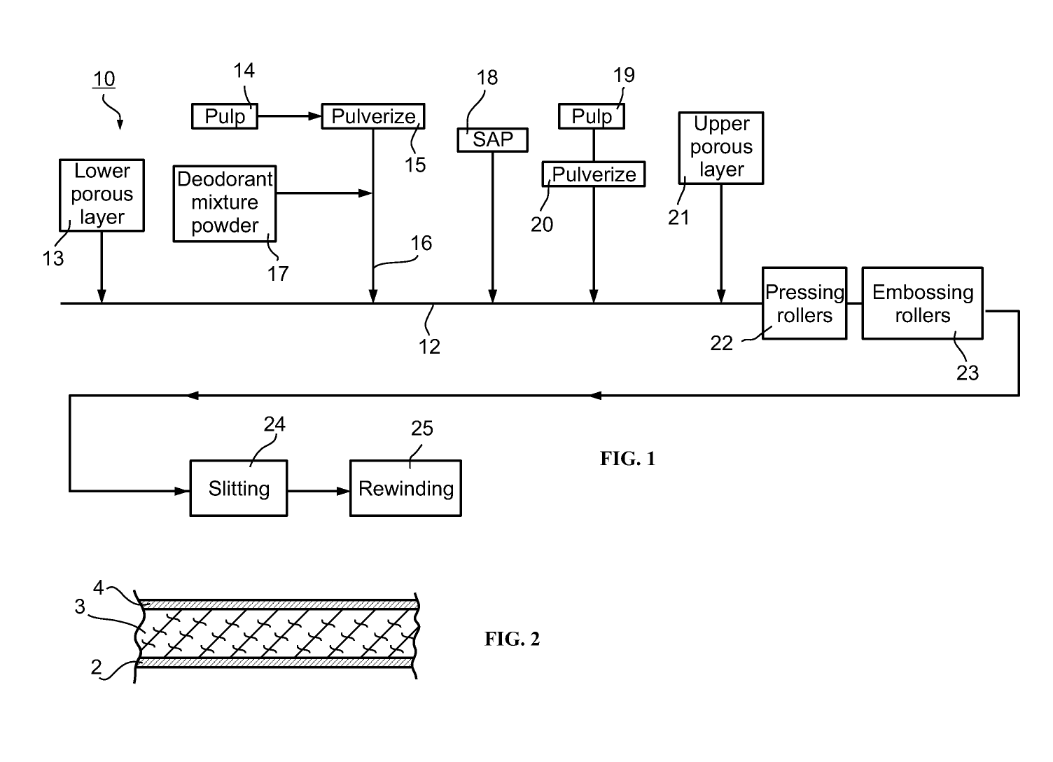

### The Embodiment of FIGS. 1-5

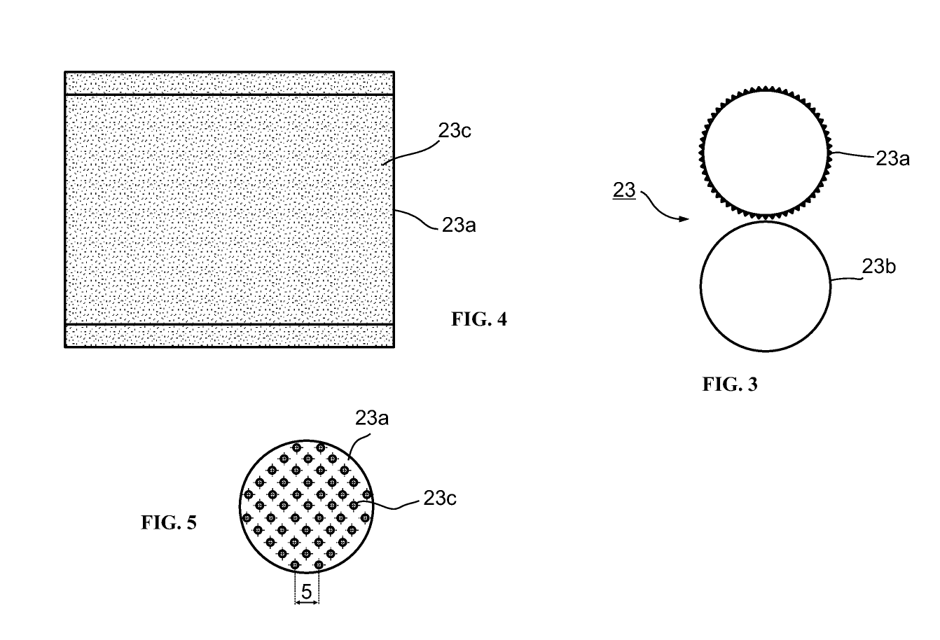

FIG. 1 schematically illustrates a production line, generally designated 10, for making an absorbent pad in accordance with the present invention. The absorbent pad made according to FIG. 1 is illustrated in FIG. 2.

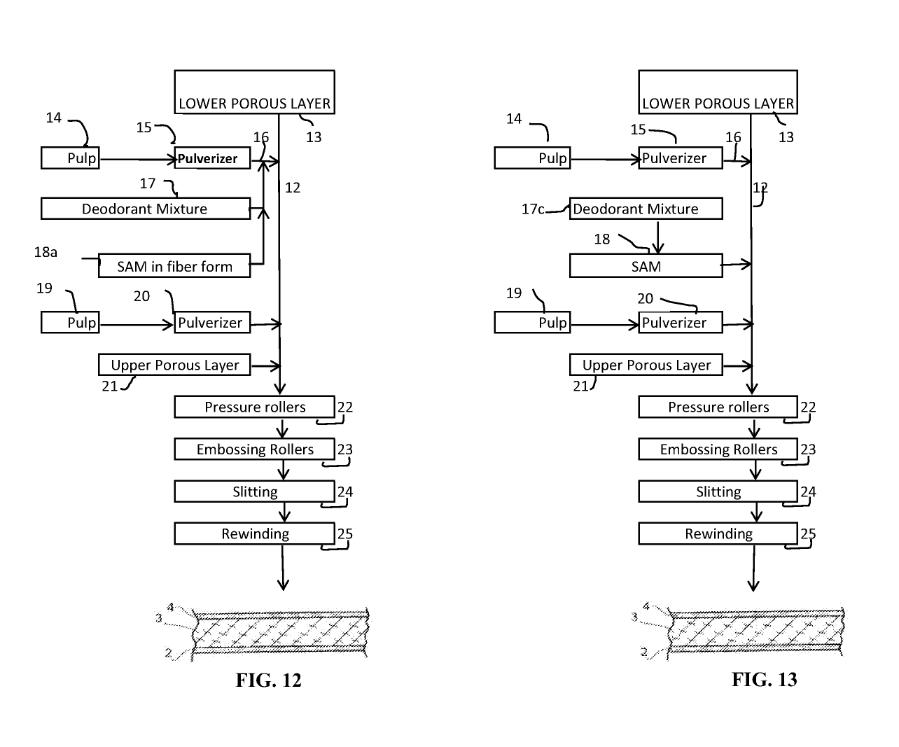

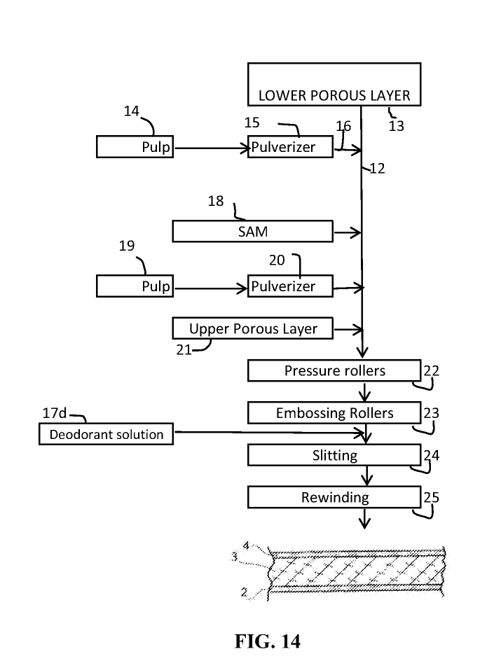

The absorbent pad produced by the production line 10 of FIG. 1 and illustrated in FIG. 2 is a three-layer pad, including a lower porous layer 2; an intermediate layer 3 of an absorbent pulverized pulp; and an upper porous layer 4. As will be described more particularly below, the three-layer absorbent pad of FIGS. 1 and 2 may be used alone, or it may be used as a core for producing an absorbent pad of more than three layers, such as the five-layer absorbent pad of FIGS. 6 and 7.

Thus, the production line 10 shown in FIG. 1 includes a web 12 moved through a number of stations at which various materials are applied thereto and processed thereon to produce the three-layer pad of FIG. 2.

The first station is shown at 13, whereat a porous layer is deposited on web 12 to form the lower porous layer 2 in the three-layer pad of FIG. 2. This lower porous layer 2 may be an unwoven paper tissue layer, or a porous plastic film such as polyethylene formed with micro-perforations.

Pulp 14 is pulverized at 15 and is separated into cellulose fibers and applied via a vacuum conveyor 16 onto the porous layer 2 on web 12. While the fibers move through the vacuum conveyor, a quantity of a deodorant mixture powder 17 is added to the fibers so as to be mixed therewith when deposited at 16 on the porous layer 2 on web 12.

The deodorant mixture powder 17 includes a deodorant agent, an anti-inflammatory agent, and/or also a perfume fragrance. The deodorant agent is preferably a talc powder, but may also be another suitable deodorant material such as triclosan. The anti-inflammatory agent may be included to prevent skin erythema. A preferred material is zinc oxide, but can also be another anti-inflammatory agent, such as urea, or allantoin or aloe vera extract. The perfume fragrance is preferably included to mask perspiration, urine, or other odors when the absorbent pad is used by a person.

The deodorant mixture powder 17, including one or more of the foregoing materials, may generally be present from 5 to 45 percent by weight, preferably about 20 percent, of the intermediate absorbent layer 3. The deodorant agent may be present from 60 to 80 percent, preferably about 75 percent, by weight, of the deodorant mixture powder; the anti-inflammatory agent may be present from 20-30%, preferably about 23%, of the deodorant mixture powder; and the perfume fragrance may be present, from about 1 to 5 percent by weight, preferably about 2 percent, of the deodorant mixture powder.

Following the deposition of the pulverized pulp 15 mixed with the deodorant mixture powder 17, a super absorbent material (SAM) 18 is deposited (optionally with odor control). Such materials are usually well-known polymers having the capability of absorbing and retaining extremely large amounts of a liquid relative to their own mass. The water absorbing polymers, classified as hydrogels, absorb aqueous solutions through hydrogen bonding with the water molecule. Preferably, the SAM material is a cross-linked sodium polyacrylate polymer made from the pulverization of acrylic acid blended with sodium hydroxide in the presence of an initiator. It will be appreciated, however, that other materials may be used to make the super absorbent polymer, such as polyacrylamide copolymer, starch grafted copolymer of polyacrylonitrile, or any of the other materials described in U.S. Pat. No. 7,025,198.

Following the deposition of the SAM material 18, another quantity of pulp 19 pulverized at 20 is applied, and then the upper porous layer 4 is applied at station 21. Preferably, the upper porous layer 4 is of the same material as the lower porous layer 2.

After the foregoing layers have been applied, the so-formed pad is passed through pressure rollers 22, embossing rollers 23, and slitting rollers 24, which trim the core edges or slit the core itself into several pieces according to the core width before being rewound on a roll as shown at 25.

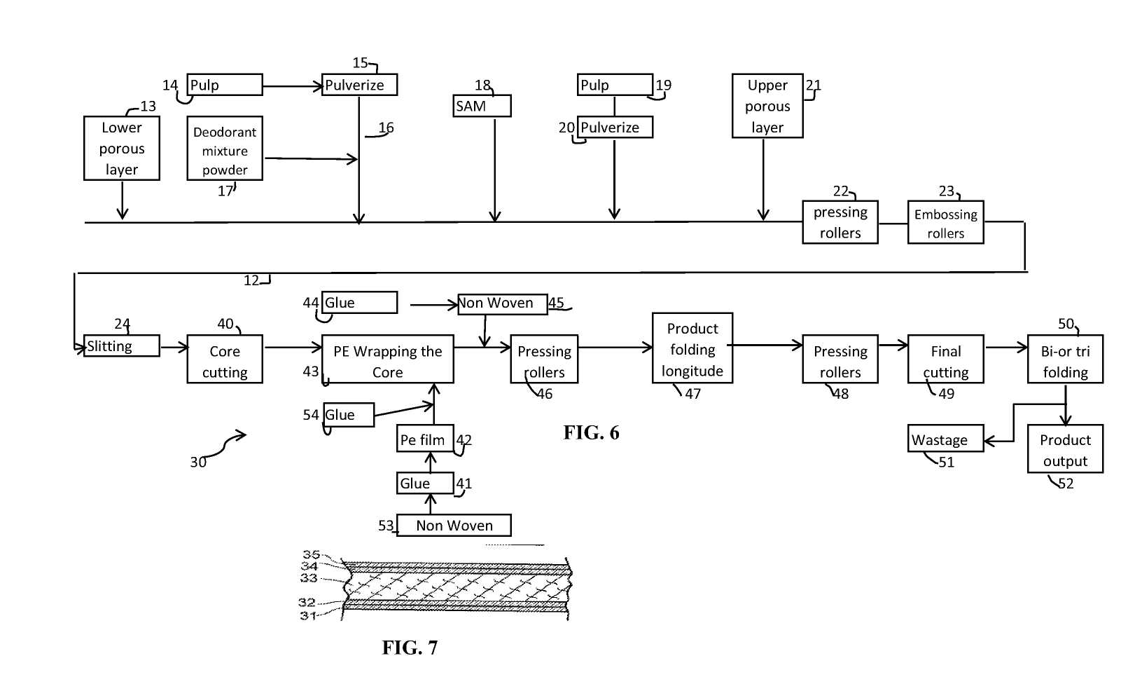

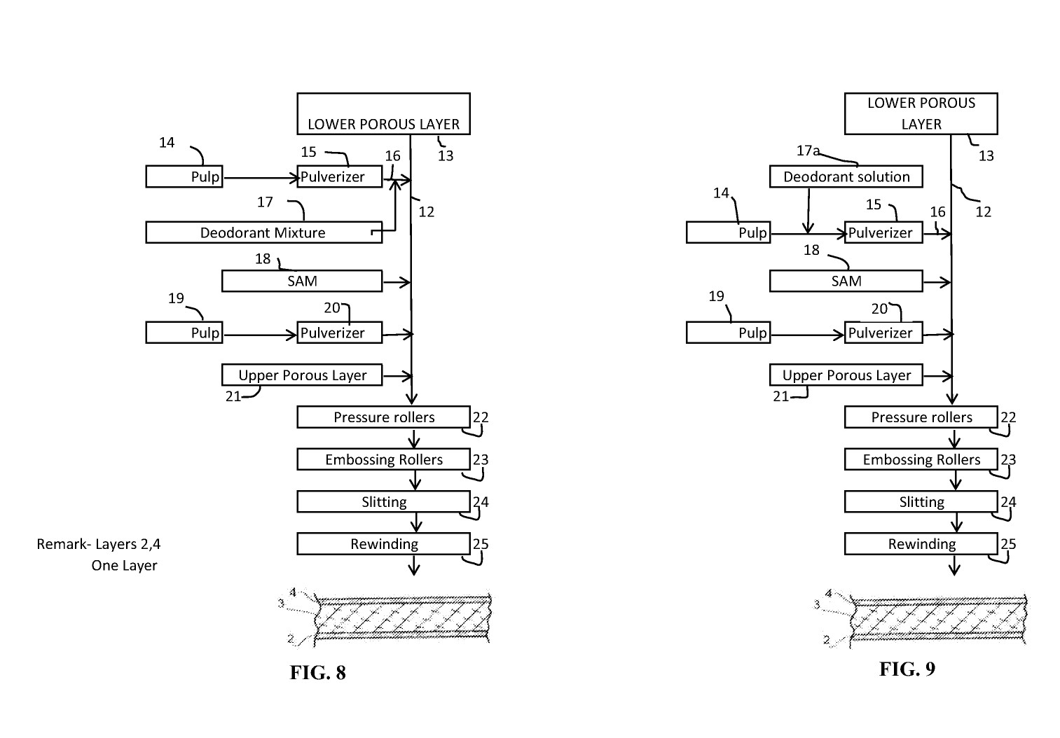

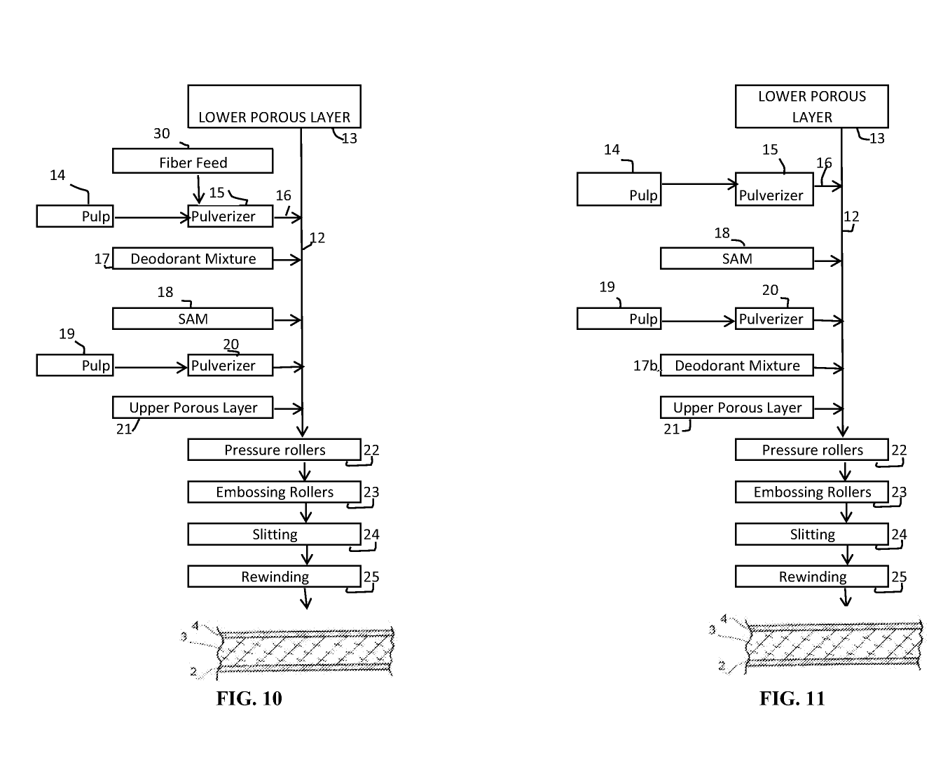

Pressure rollers 22 may be a pair of calendar rollers that bond the various layers together by pressure. Embossing rollers 23 may be a second pair of calendar rollers with embossing patterns on them to emboss the web as desired. FIGS. 3-5 illustrate one construction of embossing rollers, therein designated 23a, 23b, that may be used. As shown particularly in FIGS. 4 and 5, such embossing rollers include semi-cylindrical (or semi-spherical) projections, shown at 23c (FIG. 5) projecting from the outer surfaces of both rollers to produce semi-cylindrical (or semi-spherical) embossments in the three-layer pad shown in FIG. 2. Rollers 22 and 23 may be heated to enhance the bonding and embossing operations.

It has been found that when the foregoing method is used, wherein the absorbent body is produced by applying a first layer of a pulverized pulp to the porous under layer, then applying a layer of super-absorbent material over the first layer of pulverized pulp, and then applying a second layer of the pulverized pulp over the super-absorbent material layer, the super-absorbent material, which holds most of the absorbed liquid, is further removed from the upper surface of the pad in contact with the subject's skin (or other object from which the moisture is being absorbed), and thereby presents a drier feel or appearance to the subject (or to the article product displayed by the absorbent pad). In addition, the super-absorbent material can be selectively applied only to select locations (i.e., not to the complete surface), of the porous under layer, enabling a substantial saving to be made in the quantity of the super-absorbent material used for any particular application, and thereby substantially lower the cost for the product.

As indicated above, the absorbent pad illustrated in FIG. 2 may be used as a separate absorbent pad for certain applications, in which the pulverized pulp and SAM material in the intermediate layer 3 effectively absorb large quantities of liquids, while the deodorant powder mixture 17, added to the pulverized pulp of the intermediate layer 3, effectively prevent skin erythema and reduce odors produced during the use of the absorbent pad by a person, while the perfume fragrance masks perspiration or other (e.g. urine) odors.

### The Embodiments of FIGS. 6 and 7

FIG. 6 illustrates a production line, generally designated 30, for a method of making an absorbent pad of a larger number of layers (5-layers in the example), in which the absorbent pad of FIG. 2 constitutes the core; and FIG. 7 illustrates such an absorbent pad but further including an extra backing layer 31, and an extra overlying non-woven layer 35, constructed according to FIG. 6. The core of the FIG. 7 pad may be made off-line according to the block diagram of FIG. 1; preferably, however, it is part of an inline production method as illustrated in FIG. 6.

Thus, as shown in FIG. 7, the absorbent pad produced by the production line of FIG. 6 is a five-layer pad, rather than a three-layer pad as shown in FIG. 2. The five-layer pad of FIG. 7 includes a backing layer 31 of a non-porous plastic film, such as polyethylene; a lower porous layer 32 of the material corresponding to porous layer 2 in FIG. 3; an intermediate layer 33 including the paper pulp and super absorbent material (SAM), and also the deodorant powder mixture, corresponding to intermediate layer 3 in FIG. 2; an upper porous layer 34 of the same material corresponding to porous layer 4 in FIG. 2; and a top layer 35 of non woven porous material like spunbond or thermobond fibers.

The first part of the inline production process illustrated in FIG. 6 is substantially the same as line 10 in FIG. 1, and similarly applies the lower porous layer 32 in FIG. 7, the intermediate absorbent layer 33 and the upper porous layer 34, which layers correspond to layers 2, 3 and 4 in the three-layer pad illustrated in FIG. 2.

The absorbent pad illustrated in FIGS. 6 and 7 includes the two previously mentioned additional layers, namely backing layer 31, underlying the lower porous layer 32, and constituted of a non-porous plastic film, such as of polyethylene; and an upper non-woven fabric layer 35 overlying porous layer 34. The upper non-woven fabric layer 35 is provided primarily to produce a soft or comfortable feel to the absorbent pad. It may also be made of the same material as porous layers 32 and 34.

In some cases it may be desirable also to include such a non-woven layer (corresponding to layer 35) also as a bottom layer to produce such a soft or comfortable feel to the skin in contact with the pad.

The first part of the production line in FIG. 6, up to the slitting operation at 24, is substantially the same as described above with respect to FIG. 1, and therefore corresponding parts are correspondingly numbered. During this first part of the production line, a three layer absorbent pad is produced, constituted of layers 32, 33 and 34 in FIG. 7, corresponding to the three layers 2, 3 and 4, of the absorbent pad of FIG. 2.

The slitting operation 24 in FIG. 6 merely trims the edges of the pad, whereas the cutting operation 40 cuts the core into pieces of the desirable width and length. A layer of glue 41 is then applied to the non-porous plastic film 42, such as of polyethylene, and the non-woven fabric layer 31 is applied at 53 to the backing layer 32, as shown in FIG. 7.

### Some Variations in Making the Absorbent Pad of FIGS. 1-5

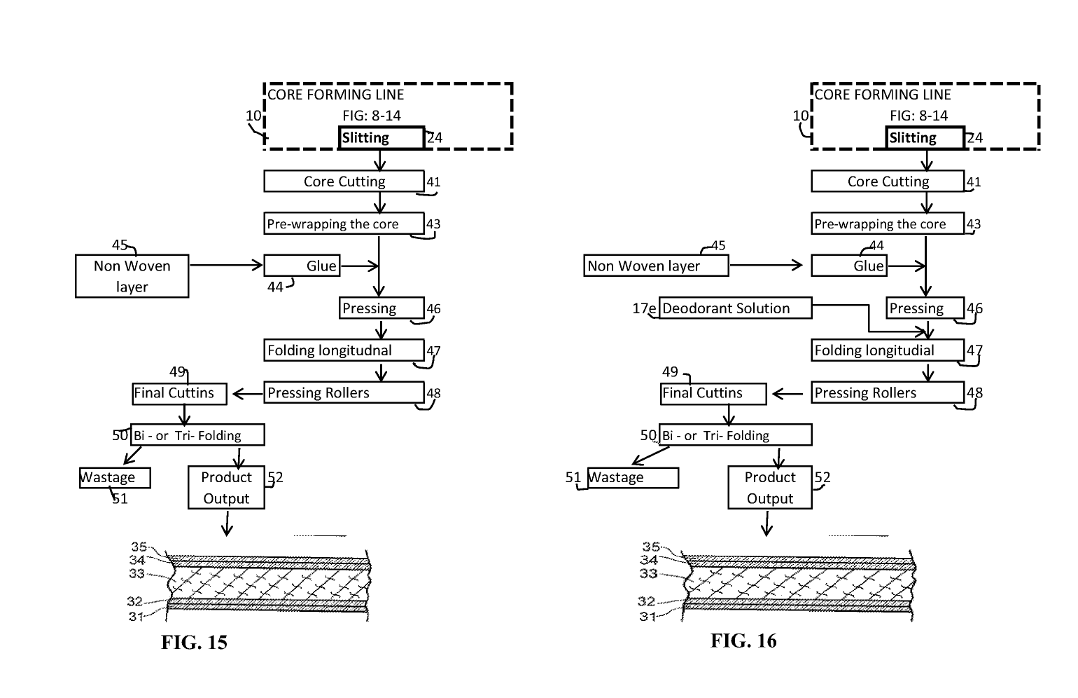

FIG. 8 is a flow chart illustrating the sequence of operations in the production line of FIG. 1. To facilitate understanding, the various blocks in the flow chart of FIG. 8 are identified by the same reference numerals as the blocks of FIG. 1. The output of the sequence of operations illustrated in FIG. 8 is an absorbent pad having the porous under layer 2, the porous upper layer 4, and the absorbent body 3 bonded to and between the two porous layers.

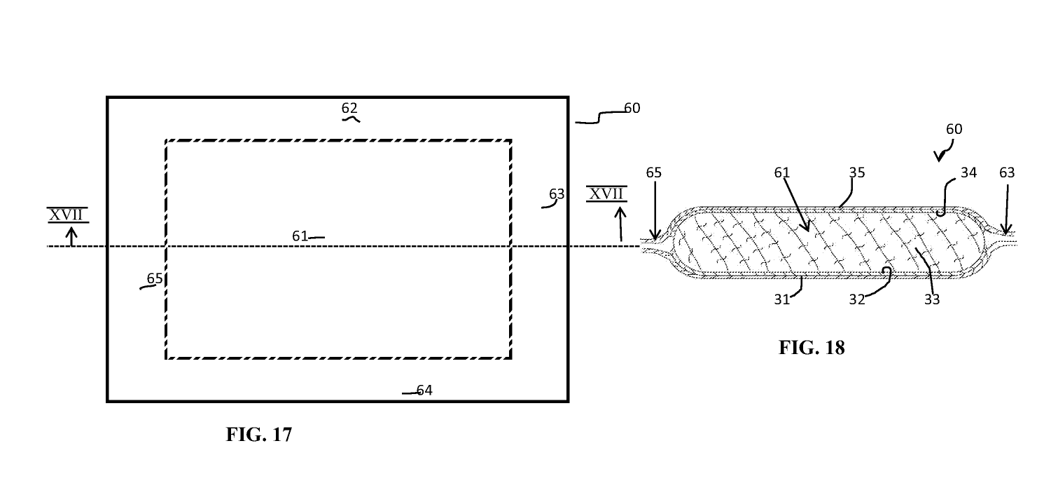

FIG. 9 is a flow chart similar to that of FIG. 8, but wherein the deodorant mixture, represented by block 17a, is applied to the pulp 14 before pulverized in the pulverizer 15 and applied to the lower porous layer 2 on web 12. In all other respects, the flow chart of FIG. 9 is the same as described above with respect to FIGS. 8 and 1, and therefore the corresponding blocks are identified with the same reference numerals.

FIG. 10 illustrates a variation in the flow chart of FIG. 8, wherein fibers, identified by box 30, is fed to the pulverized pulp before applied to the porous under layer 12. The fibers indicated by block 30 in FIG. 10 are preferably of polypropylene fibers or viscose fibers, of about 6-12 mm in length, and applied to constitute between 5-15% of the pulp mixture of the respective porous under layer 12. In all other respects, the flow chart illustrated in FIG. 10 is similar to that of FIG. 8, and its corresponding blocks are identified by the same reference numerals. The addition of the fibers, as indicated by block 30, adds significantly to the overall strength of the produced absorbent pad.

FIG. 11 illustrates a further variation wherein the deodorant mixture, represented by block 17 in FIG. 8, is applied to the porous under layer 12 on web 2 following the application of the second pulverized pulp. This is indicated in FIG. 11 by the deodorant mixture block 17b applying the deodorant mixture to the porous under layer 12 after the second pulverized pulp, indicated by blocks 19 and 20, is applied to that porous under layer. In all other respects, the flow chart of FIG. 11 is similar to that of FIG. 8, and therefore the corresponding blocks are identified with the same reference numerals.

FIG. 12 is a flow chart similar to that of FIG. 8, but wherein the super-absorbent material is itself a polymer in fiber form, as indicated by block 18a in FIG. 12. Accordingly, the super-absorbent material, being in fiber form, adds to the mechanical strength of the produced absorbent pad, similar to the process illustrated in the flow chart of FIG. 10.

FIG. 13 illustrates a variation in the process of FIG. 8, in that the deodorant mixture, therein indicated by block 17c, is added to the super-absorbent material, rather than to the pulverized pulp, before the super-absorbent material is applied to the porous under layer. In all other respects, the process illustrated in FIG. 13 is similar to that of FIG. 8, and therefore the corresponding boxes have been identified by the same reference numerals.

FIG. 14 illustrates a further variation wherein the deodorant mixture (or solution) 17d is added after the embossing rollers 23.

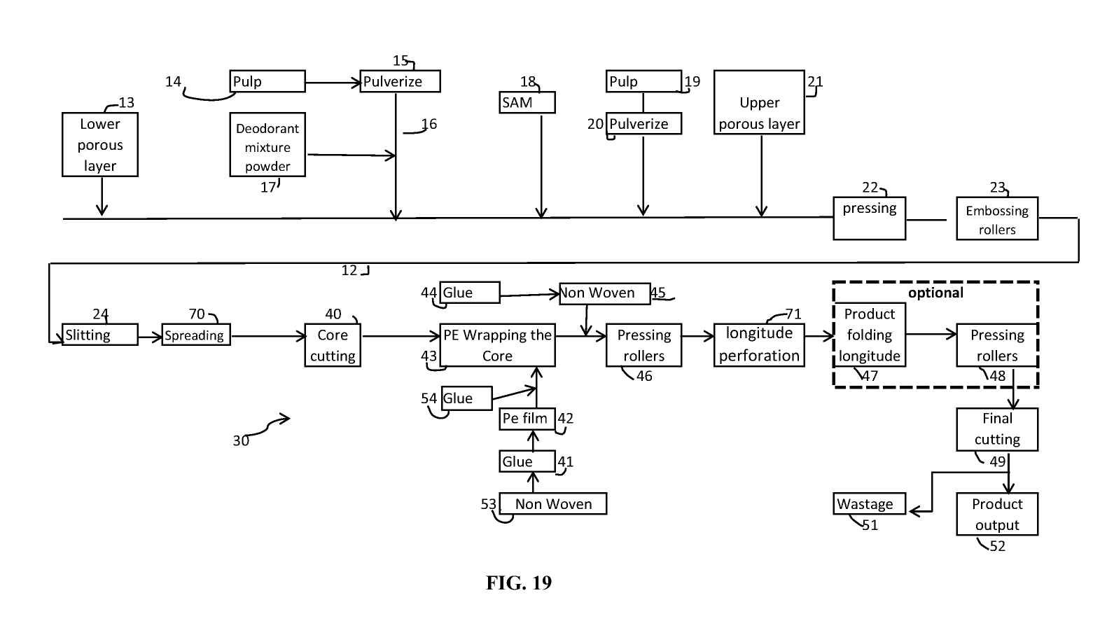

FIG. 15 is a flow chart illustrating the sequence of steps in the production line illustrated in FIG. 6. As indicated earlier, the three-part absorbent pad described above with respect to FIGS. 1-5 and 8-14 may be produced separately, transported to another location if desired, and then completed to produce the five-part absorbent pad of FIG. 6. Preferably, however, the production line illustrated in FIGS. 1-5 and 8-14 also includes in-line the various further operations illustrated in FIGS. 6 and 15.

Thus, the flow chart illustrated in FIG. 15 includes all the steps, represented by block 10 in FIG. 1, for producing the three-part absorbent pad illustrated in FIG. 2, except that the rewinding step 25 in FIG. 1 is omitted, so that the last operation in producing the three-part absorbent pad ends with the slitting operation 24, as shown in FIG. 6. The same production line, in this case, thus also includes the other operations generally identified by reference numeral in FIG. 6, including the specific operations labeled 41-52 in FIG. 6. FIG. 15 identifies these latter operations by the same numerals as used in FIG. 6.

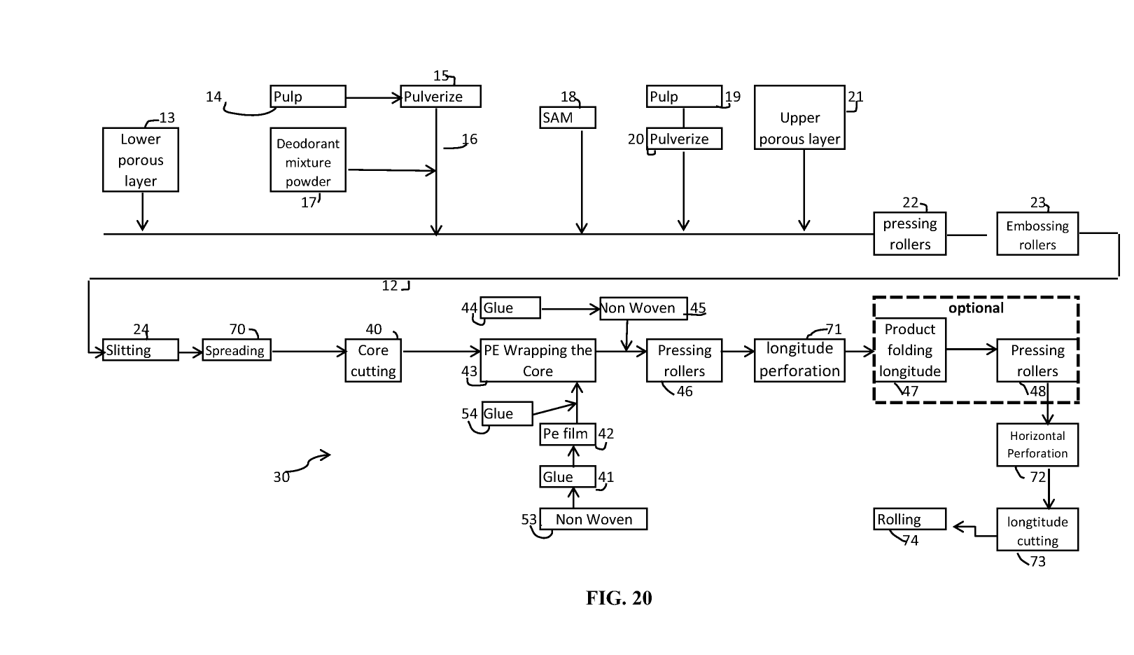

FIG. 16 illustrates a modification wherein in the in-line operation therein illustrated, the deodorant mixture (or solution) may be added, as described in any of the variations of FIG. 8-14, or following the pressing roller step 46, as indicated by block 17e in FIG. 15.

### Another Example of an Absorbent Pad Construction

FIGS. 17 and 18 illustrate another example of the construction of an absorbent pad in accordance with the present invention. In this example, the absorbent pad, therein generally designated 60, is of the five-layer construction illustrated in FIG. 7 and is made according to the production line of FIG. 6 or flow chart of FIG. 15.

As shown in FIGS. 17 and 18, backing layer 31 and the top porous layer 35 are preferably of larger dimensions than the core pad constituted of layers 32, 33 and 34, so that the core 61 of the pad is circumscribed on all four sides by borders 62-65 consisting only of layers 31 and 35 bonded together on the four opposite sides of the 5-layer pad 60.

After the 3-layer absorbent pad 61 has been produced, it is passed between a pair of pressing rollers 46 (FIG. 15 or 16) for compacting them, then folded as shown at 47, pressed as shown at 48, and finally cut as shown at 49. The so-prepared pad may then be bi-folded or tri-folded, as shown at 50. The wastage is rejected, as shown at 51, whereas the produced 5-layer pad appears as a final product at 52.

The pulp used is preferably a commercially available pulp having a humidity larger than 5 percent, preferably 7-10 percent. The pressure rollers need not be heated so that the bonding is affected by pressure alone. However, the embossing rollers shown at 23a and 23b in FIGS. 3-5, which may be heated, are used to enhance the embossing operation.

The produced absorbent pad 60 thus includes a non-porous backing layer 61, a porous tissue layer 32, an absorbent body 33, an upper tissue layer 34, underlying the 3-part form illustrated in FIG. 2, and a non-woven top layer 35 to improve the “feel” of the absorbent pad. As indicated earlier, it may also include a non-woven bottom layer 31 to be in contact with the subject's skin. The absorbent body 33 is of smaller dimensions, both lengthwise and widthwise, from the two lower layers 31, 32 and upper layers 34, 35, to occupy only the space needed for the absorbent layer, e.g. when the absorbent pad is used as a soak pad for soaking liquids from meat and fish packaging. Such a construction is easily producible since the super-absorbent material is separately applied, and therefore can be applied at selective locations according to the particular use of the absorbent pad, thereby enabling substantial savings to be made in such absorbent pads.

### Methods of Making Absorbent Pad of FIGS. 17 and 18

FIGS. 19-22 illustrate various methods of making absorbent pads in a continuous manner according to the construction of FIGS. 17 and 18. The methods illustrated in FIGS. 19-22 are basically the same as that illustrated in FIG. 1 through the slitting operation 24 to produce the three-layer pad illustrated in FIG. 2 as a continuous strip. As described above, the three-layer pad illustrated in FIG. 2 may be used as a separate absorbent pad for certain applications, may also be used as the core of a five-layer absorbent pad as described above with respect to FIGS. 6 and 7. Where the absorbent pads to be produced are of the construction of FIGS. 17 and 18, namely individual pads each circumscribed on all four sides by borders 62-65 consisting only of layers 31 and 35 bonded together on the four opposite sides of the five-layer pad, instead of the rewinding operation 25 illustrated in FIG. 1, the production line would include the further operations as illustrated in FIGS. 19-22.

To facilitate understanding, the operations illustrated in FIGS. 19-22 which generally correspond to those illustrated in FIGS. 1 and 6, are identified by the same reference numerals, the additional operations performed in the method illustrated in FIGS. 19-22 being identified by reference numerals starting with “70”.

Thus, the slitting operation 24 illustrated in FIG. 19 slits the continuous three-layer absorbent pad (as illustrated in FIG. 2) along a plurality of longitudinal slit lines (e.g. four slit lines to define five separate strips). The separate strips are then spread apart at station 70, followed by the various operations illustrated in FIG. 7, but modified as illustrated in FIG. 19.

Thus, in the method of FIG. 19, the core cutting operation 40 cuts the three-layer core strips along transverse lines before the two non-woven outer layers 31 and 35 (FIG. 7) are applied. After these layers are applied, as described above with respect to FIG. 7, the five-layer strip is then formed with longitudinal perforations at station 71 so that the product output at station 52 will consist of, e.g. five longitudinal strips of the absorbent pads of FIGS. 17 and 18 folded longitudinally on top of each other and connected together by weakened lines defined by the longitudinal perforations produced at station 71 in FIG. 19.

The method illustrated in FIG. 20 is similar to that of FIG. 19 as described above, except that in addition to the longitudinal perforation step 71, a horizontal perforation step 72 is also performed, followed by a longitudinal cutting step 73, and a rolling step 74, such that the product output will be individual rolls of longitudinal strips of absorbent pads, as illustrated in FIGS. 17 and 18, each including transverse lines of perforations to facilitate individual absorbent pads to be separated from the respective roll.

FIG. 21 is similar to that of FIG. 20, except that operations 47 and 48 in FIG. 20 have been omitted; and FIG. 22 illustrates a method similar to that of FIG. 19 in which the product folding operation 47 and pressing rollers operation 48 have also been omitted.

While the invention has been described with respect to several preferred embodiments, it will be appreciated that these are set forth merely for purposes of example, and that many other variations, modifications and applications of the invention may be made.

What is claimed is:

1. A method of making an absorbent pad comprising a porous under layer, a porous upper layer, and an absorbent body including a pulverized pulp and a super-absorbent material bonded to and between the porous layers; characterized in that the absorbent body is applied between the porous layers before bonding thereto by:
applying a first layer of pulverized pulp over the porous under layer;
then applying a layer of the super-absorbent material over the first layer of pulverized pulp;
and then applying a second layer of the pulverized pulp over the super-absorbent layer.

2. The method according to claim 1, wherein a deodorant material is mixed with the pulverized pulp of the first layer before applied over the porous under layer, or with the pulverized pulp of the second layer before applied over the super-absorbent layer.

3. The method according to claim 1, wherein fibers are mixed with the pulverized pulp of the first layer before applied over the porous under layer, or with the pulverized pulp of the second layer before applied over the super-absorbent layer.

4. The method according to claim 3, wherein the fibers are selected from the group consisting of polypropylene fibers, viscose fibers and fibers of a superabsorbent material.

5. The method according to claim 3, wherein the fibers constitute 5-15% by weight of the respective layer.

6. The method according to claim 3, wherein the fibers are of 6-12 mm in length.

7. The method according to claim 1, wherein the super-absorbent material is in the form of fibers of a polymeric material.

8. The method according to claim 1, wherein the super-absorbent material is or includes a deodorant.

9. The method according to claim 1, wherein the absorbent body has an initial water content of 7-10% before bonded to and between the porous layers.

10. The method according to claim 1, further comprising:
applying a non-porous backing layer over the surface of the porous under layer opposite to that bonded to the absorbent body;
and applying a non-woven fabric layer over the surface of the porous upper layer opposite to that bonded to the absorbent body.

11. The method according to claim 10, wherein the absorbent body is of smaller width and length than the non-porous backing layer and the non-woven fabric layer.

12. A method of making an absorbent pad comprising a porous under layer, a porous upper layer, and an absorbent body including a pulverized pulp and a super-absorbent material bonded to and between the porous layers; characterized in that the absorbent body also includes a deodorant composition.

13. The method according to claim 12, wherein fibers are mixed with the pulverized pulp before applied to the porous under layer.

14. The method according to claim 13, wherein the fibers are selected from the group consisting of polypropylene fibers or viscose fibers.

15. The method according to claim 13, wherein the fibers constitute 5-15% by weight of the pulverized pulp in the respective layer.

16. The method according to claim 13, wherein the fibers are of 6-12 mm in length.

17. The method according to claim 13, wherein the super-absorbent material is in the form of fibers of a polymeric material.

18. The method according to claim 13, wherein the absorbent body has an initial water content of 7-10% before bonded to and between the porous layers.

19. The method according to claim 13, further comprising:
applying a non-porous backing layer over the surface of the porous under layer opposite to that bonded to the absorbent body;
and applying a non-woven fabric layer over the surface of the porous upper layer opposite to that bonded to the absorbent body.

20. An absorbent pad of a plurality of layers bonded together, comprising a porous under layer; a porous upper layer; and an absorbent body bonded to and between said porous layers; characterized in that the absorbent body includes:
a first layer of a pulverized pulp applied over said porous layer;
an intermediate layer of a super-absorbent material applied over the first layer of pulverized pulp;
and a second layer of a pulverized pulp over the super-absorbent layer.

21. The absorbent pad according to claim 20, wherein at least one of said layers of a pulverized pulp includes a deodorant material.

22. The absorbent pad according to claim 20, wherein at least one of said layers of a pulverized pulp includes fibers effective to increase the strength of the absorbent pad.

23. The absorbent pad according to claim 22, wherein the fibers are selected from the group consisting of polypropylene fibers, viscose fibers and fibers of a superabsorbent material.

24. The absorbent pad according to claim 22, wherein the fibers constitute 5-15% by weight of the pulverized pulp in the respective layer.

25. The absorbent pad according to claim 22, wherein the fibers are of 6-12 mm in length.

26. The absorbent pad according to claim 20, wherein the super-absorbent material is in the form of fibers of a polymeric material.

27. The absorbent pad according to claim 20, wherein the super-absorbent material is or includes a deodorant.

28. The absorbent pad according to claim 20, wherein the absorbent body has an initial water content of 7-10% before bonded to and between the porous layers.
